(19) United States
(12) Patent Application Publication
(10) Pub. No.: US 2007/0100280 A1
(43) Pub. Date: May 3, 2007

(54) Catheter with balloon material having visual marker

(76) Inventors: Leonard A. van Sloten, Groningen (NL); Cornelius van Wee, Drachten (NL)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/590,538

(22) Filed: Oct. 31, 2006

Related U.S. Application Data
(63) Continuation-in-part of application No. 11/515,528, filed on Sep. 5, 2006.
Application No. 11/515,528 is a continuation-in-part of application No. 11/119,259, filed on Apr. 29, 2005.
Application No. 11/119,259 is a continuation-in-part of application No. 11/095,948, filed on Mar. 31, 2005.

(51) Int. Cl. A61M 29/00; A61M 31/00; A61M 37/00

(52) U.S. Cl. 604/103.1

(57) ABSTRACT

A balloon catheter may have a flexible shaft extending between a proximal and distal end, with a hub affixed to the proximal end, and a balloon made of material that is imprinted with one or more visual markers. The visual marker(s) indicate and confirm specific information about attribute(s) of that individual product.

### CROSS-REFERENCE To RELATED APPLICATION

The present application is a continuation-in-part of Ser. No. 11/515,528 entitled “Catheter With Balloon Having Visual Marker” filed Sep. 5, 2006, which is a continuation-in-part of Ser. No. 11/119,259 entitled “Esophageal Balloon Catheter With Visual Marker” filed Apr. 29, 2005, which is a continuation-in-part of Ser. No. 11/095,948 entitled “Esophageal Catheter With Asymmetrical Balloon” filed Mar. 31, 2005.

### BACKGROUND AND SUMMARY OF THE INVENTION

1. Technical Background

The present invention relates generally to medical devices, and more particularly to a catheter with a balloon having at least one visual marker which indicates product information.

2. Discussion

There are many different kinds and types of balloon catheters, including for example angioplasty catheters, stent delivery system catheters, etc.

By way of example, the present invention will be described in relation to a typical balloon catheter. Also, some more specific examples will be described, including an angioplasty catheter, and a balloon catheter for use with an endoscope. However, it should be understood that the present invention relates to any balloon catheter having the features recited in any one of the following claims, and is not limited to any particular treatment such as angioplasty, esophageal use or use with an endoscope, or the particular example embodiments described below.

In general, balloon catheters according to the present invention may have one or more of the following features: a substantially inelastic balloon made of material that is imprinted with at least one marker indicating information about attributes of that particular product.

In greater detail, one or more visual markers may be provided on the balloon material or in the balloon material itself. Alternatively, the entire balloon may have a specific color that confirms certain product information. If the marker(s) are color-coded, a marker of a particular color may indicate certain properties, such that the catheter balloon is of a particular size. This visual indicator enables a physician to quickly confirm that the desired size balloon has been selected for use. Visual markers may also be made of different sizes or patterns, to indicate balloon catheter properties.

Balloon catheters often have a relatively flexible tubular shaft of a certain length, which defines one or more tubular passages or “lumens” extending through part or all of the catheter shaft, and an inflatable balloon attached near one end of the shaft. This end of the catheter where the balloon is located is customarily referred to as the “distal” end, while the other end is called the “proximal” end. The proximal end of the shaft is generally coupled to a hub, which defines an inflation port for connection to an inflator for selectively applying pressure to a fluid inflation medium, thus inflating the balloon. Structurally, the inflation port leads to an inflation lumen defined by the shaft, which extends to and communicates with the interior of the balloon, for the purpose of selectively inflating and deflating the balloon.

When a catheter includes a lumen adapted to slidingly receive a guidewire, it is referred to as a “guidewire lumen,” and it will generally have a proximal and distal “guidewire port.” The distal guidewire port is often at or near the catheter shaft distal end.

A guidewire has a flexible wire-like structure extending from a proximal end to a distal end. The guidewire will usually be of a size selected to fit into and slide within a corresponding guidewire lumen of a catheter.

If a balloon catheter includes a hub affixed to the catheter shaft proximal end, the hub may serve a variety of functions. These functions may include providing a handle for manipulating the catheter, and/or defining proximal port(s) communicating with lumen(s) defined by the catheter shaft. When there is a guidewire lumen defined by a catheter shaft, its proximal guidewire port may be defined by a proximal hub, referred to as an “over-the-wire” catheter; or the proximal guidewire port may be located at some point along the sidewall of the catheter shaft, referred to as a “rapid exchange” catheter.

When a catheter has no guidewire lumen, but instead has a flexible wire or wire-like distal extension affixed to the catheter, it may be referred to as a “fixed wire” catheter. Whether a particular catheter has a guidewire lumen or has a fixed-wire design, the guidewire or fixed-wire is intended to allow the catheter to more easily select and steer along a desired path.

In a fixed wire balloon catheter, a wire or wire-like structure may simply be attached to the distal end of the balloon catheter. Alternately, a flexible wire or wire-like structure may be affixed to the proximal hub, extending from the proximal end of the catheter, though the shaft and the balloon (perhaps in a dedicated lumen), and may extend a relatively short distance distal of the balloon. In another possible configuration, a distal extension of an inner body of the catheter shaft may serve as a “fixed wire” guiding element.

Balloon catheters may also be used to deliver or deploy medical devices, including stents or drug-coated stents, which are mesh scaffolds for holding open a body passage of a patient.

Each particular type of balloon catheter may also be available with some options, including various sizes and lengths, or having different strengths or compliance pressure curves.

Because many balloon catheters are relatively small, and may have translucent components, it may be desirable to provide the balloon itself with a visual indicator that confirms specific information about that particular product. For example, the balloon itself may have a color or a visual marker or a pattern, even specific characters. Such indicators may confirm the size of the balloon, or that the balloon has “high strength” or is a “compliant” balloon, etc.

Visual markers may assist a physician to accurately position the balloon. In the case where a balloon catheter is used with an endoscope, the marker may be viewed visually with the endoscope, by using the endoscopic lens to look through the balloon material of the proximal tapering portion. In other words, the physician's view is provided by an endoscope positioned proximal of the balloon, yet the physician can look through the translucent material of the balloon proximal tapering portion, and see the interior surface of a cylindrical working portion to visualize where the marker(s) is from the “inside.”

A visual marker may have any suitable shape or arrangement, including a circumferential band placed at the longitudinal center of the balloon, or a marker placed at one or both of the transitions between a central working portion and the proximal and distal tapering portions. Such markers may enable a physician to use the view through an endoscope to accurately position the balloon at the desired site for treatment, for example centered within a lesion or stricture. Of course, various combinations of these marker arrangements may be used.

Another possible option is that the markers may be combination markers, which can be seen not only with visible light using an endoscope, but also with at least one additional viewing system, such as for example x-ray fluoroscopy, magnetic resonance imaging, etc.

In the case of a balloon catheter for use with an endoscope, the balloon material may be translucent, to allow a physician to use the endoscope to look through the balloon material at the anatomy, so the physician can accurately position the balloon.

Another possible feature may be high pull strength, which may include a catheter shaft of a balloon catheter with reinforcement, such as reinforcing braid or strand(s). The resulting stronger catheter shaft will thus exhibit low longitudinal elongation under stress. Accordingly, if retraction becomes difficult, such reinforcing element(s) will tend to resist elongation of the catheter shaft.

An optional additional feature may be a balloon with a cylindrical working portion, flanked by proximal and distal tapering portions, which are in turn flanked by proximal and distal balloon legs, which are affixed to the catheter shaft.

This disclosure of the present invention will include various possible features and embodiments. However, the present invention scope as set forth in each of the claims, and is not limited to the particular arrangements described in this disclosure.

The terms “tube” and “tubular” are used in their broadest sense, to encompass any structure arranged at a radial distance around a longitudinal axis. Accordingly, the terms “tube” and “tubular” include any structure that (i) is cylindrical or not, such as for example an elliptical or polygonal cross-section, or any other regular or irregular cross-section; (ii) has a different or changing cross-section along its length; (iii) is arranged around a straight, curving, bent or discontinuous longitudinal axis; (iv) has an imperforate surface, or a periodic or other perforate, irregular or gapped surface or cross-section; (v) is spaced uniformly or irregularly, including being spaced varying radial distances from the longitudinal axis; or (vi) has any desired combination of length or cross-sectional size.

Any suitable material may be used to make the components described, including polymers, metals and other materials suitable for use with medical devices.

It is of course possible to build various kinds and designs of catheters according to the present invention, by various techniques and of various materials, to obtain the desired features. It should be noted that the present invention also relates to methods for making and using a balloon catheter, in addition to the balloon catheter itself.

These and various other objects, advantages and features of the invention will become apparent from the following description and claims, when considered in conjunction with the appended drawings. The invention will be explained in greater detail below with reference to the attached drawings of a number of examples of embodiments of the present invention.

### BRIEF DESCRIPTION OF THE DRAWINGS

FIG. 1 is a perspective view of a rapid-exchange balloon catheter;

FIG. 2 is a perspective view of an over-the-wire balloon catheter;

FIG. 3 is a perspective view of a balloon catheter;

FIG. 4 is a longitudinal cross-section view of a balloon catheter;

FIG. 5 is a longitudinal cross-section view of a balloon catheter;

FIG. 6 is a transverse cross-section view of the balloon catheter of FIG. 5; and

FIG. 7 is a partial side elevation view of a balloon catheter;

FIG. 8 is a partial cross-sectional view of a balloon catheter;

FIG. 9 is a partial cross-sectional view of a balloon catheter stent delivery system;

FIG. 10 is a transverse cross-sectional view of a balloon folded and wrapped around a catheter shaft tube; and

FIG. 11 is a partial cross-sectional view of a balloon having more than one layer and a marker in between.

### DETAILED DESCRIPTION OF THE PREFERRED EMBODIMENTS

The following description of the preferred embodiments of the present invention is merely illustrative in nature, and as such it does not limit in any way the present invention, its application, or uses. Numerous modifications may be made by those skilled in the art without departing from the true spirit and scope of the invention.

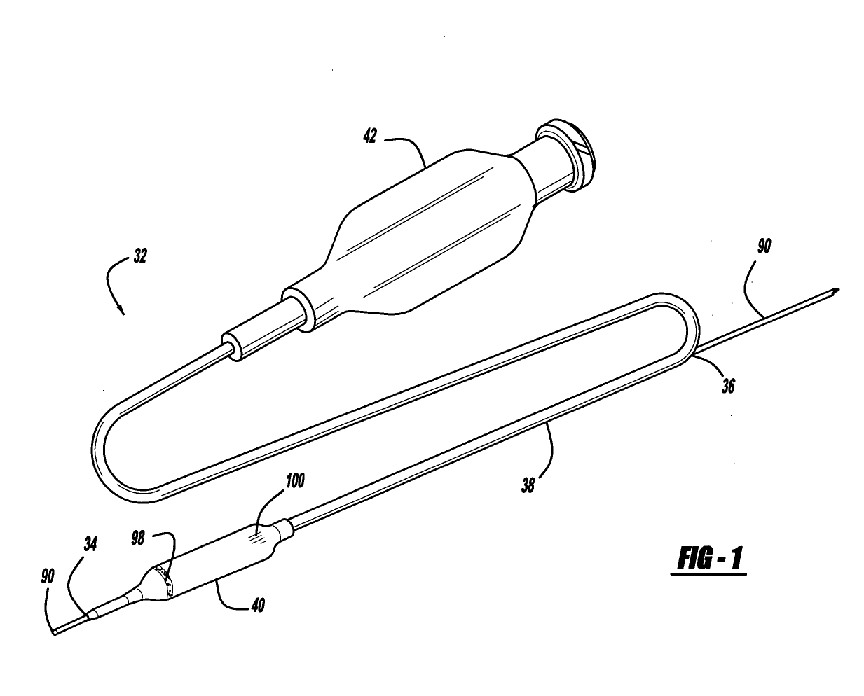

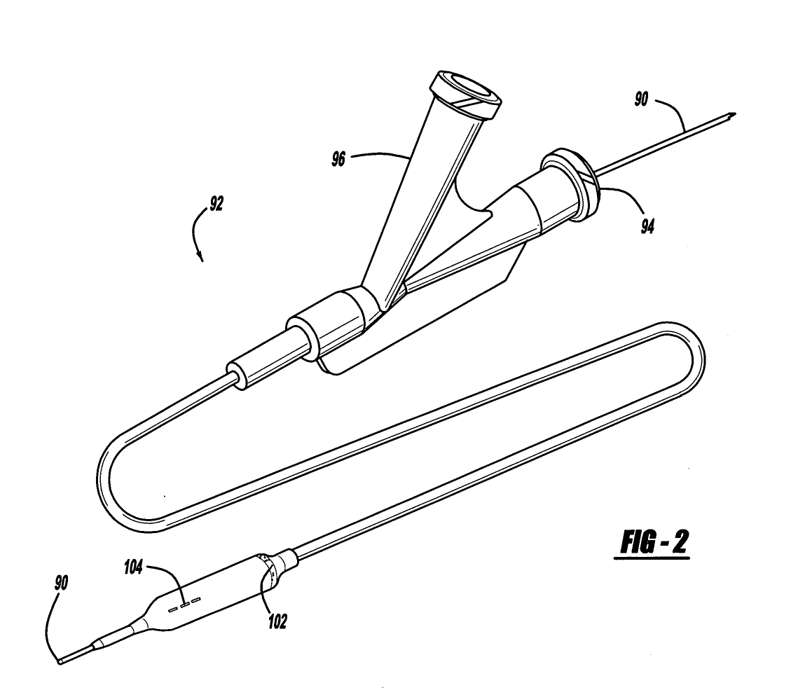

The drawings depict a variety of balloon catheters and various features. FIG. 1 shows a rapid-exchange balloon catheter 32, which defines a guidewire lumen extending from a distal guidewire port 34 at the distal end of the catheter to a proximal guidewire port 36 at a position along the shaft 38 somewhere between the balloon 40 and the hub 42. The balloon 40 is an integral, unitary piece. The balloon catheter 92 of FIG. 2 is similar, though the proximal guidewire port 94 is defined by the proximal hub 96.

Different examples of possible markers on the balloon are shown in FIGS. 1 and 2. Specifically FIG. 1 shows a first marker 98 as a circumferential stripe that may be specifically color-coded, and a second marker 100. The second marker 100 in this case is a series of three lines and a dot, which for example might signify this is a 3.5 mm balloon diameter at rated pressure.

The balloon of FIG. 2 likewise has a first marker 102 in the form of a circumferential stripe which may be color-coded, and a second marker 104 which in this case is a series of three dots.

FIG. 3 shows a balloon catheter 10 having a balloon 12, a flexible shaft 14, and a hub 16. The shaft 14 has a proximal and distal end, with the balloon 12 being attached to the shaft 14 near the distal end, and the hub 16 attached to the shaft 14 near the proximal end. A distal tip element 18 is affixed to the shaft 14 at the distal end, and a strain relief 20 is positioned at a transition between the shaft 14 and the hub 16. Balloon 12 has a cylindrical working portion 22, flanked by a proximal and distal tapering portion 24 and 26, which are in turn flanked by a proximal and distal balloon leg 28 and 30.

In addition, balloon catheter 10 has some visual markers on the material of the balloon 12. In the specific example shown in FIG. 1, the balloon 12 has a central marker 44 that encircles the longitudinal center of the balloon, as well as a pair of markers 46 and 48 which indicate the proximal and distal extent of a working portion 22 of the balloon. The visual markers may be of various sizes, colors, and arrangements, to indicate specific information about that particular product. In the example shown in FIG. 3, markers 46 and 48 are wider than central marker 44.

In contrast to the fixed-wire balloon catheters shown in FIGS. 3-6, the balloon catheters of FIGS. 1-2 and 8-10 are intended for use with a separate guidewire 90 received within a guidewire lumen.

The markers of the present invention may be coded. For example, a marker of a particular color may indicate certain properties, such that the catheter balloon is of a particular size, allowing a physician to quickly confirm that the desired size balloon has been selected for use. Visual markers may also be made of different colors, sizes or patterns, to indicate properties of an individual balloon catheter. Of course, a variety of other markers and indicators of various configurations may be used, including lines, arrows, circles, text, triangles, pointers, even digits or characters, etc.

Another possible option is that the markers may be applied or made using a variety of materials and techniques. Markers may be applied to the balloon's outer surface or inner surface, or if the balloon has more than one layer of material, the markers may be present between layers of the balloon. The marker material may be any suitable material, including ink, pigment, or paint, and may be applied using spray, adherence, shrink-wrap, applications, or extrusion technologies.

Another possible option is that the markers may be combination markers, which can be seen not only with visible light using an endoscope, but also with at least one additional viewing system, such as for example x-ray fluoroscopy, magnetic resonance imaging, etc.

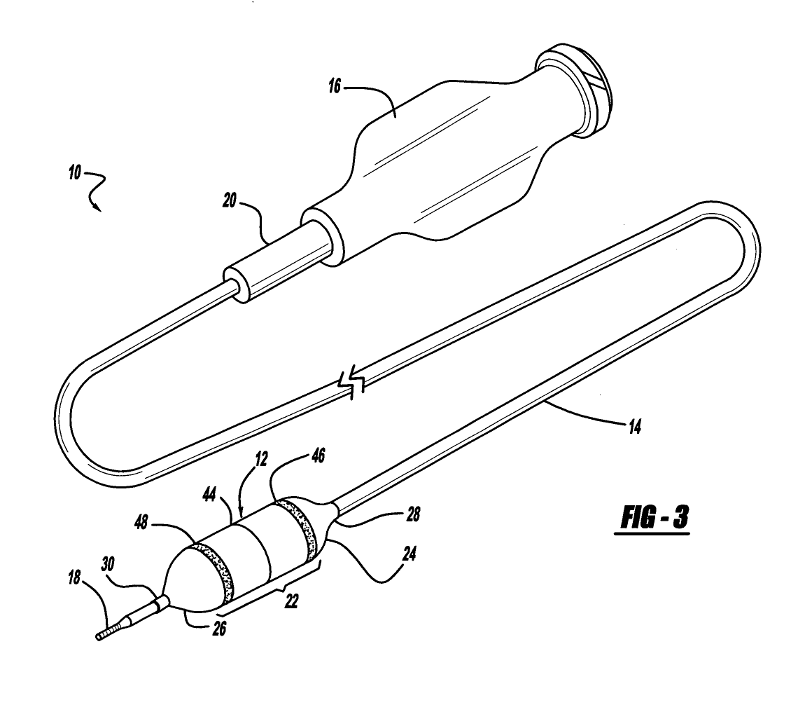

FIG. 4 shows a balloon catheter 50 having a balloon 52, a flexible shaft 54, and a hub 56. The shaft 54 has a proximal and distal end, with the balloon 52 being attached to the shaft 54 near the distal end, and the hub 56 attached to the shaft 54 near the proximal end. The hub 56 defines an inflation port in fluid communication with an inflation lumen defined by the shaft. A distal tip element 58 is affixed to the shaft 54 at the distal end, and a strain relief 60 is positioned at a transition between the shaft 54 and the hub 56. Balloon 52 has a cylindrical working portion 62, flanked by a proximal and distal tapering portion 64 and 66, which are in turn flanked by a proximal and distal balloon leg 68 and 70.

The flexible shaft 54 of FIG. 2 includes an inner member 72 and an outer tubular body 74. Inner member 72 extends from the hub 56 to the distal tip element 58, and may have high pull strength to serve as a reinforcing wire. The resulting stronger catheter shaft will thus exhibit low longitudinal elongation under stress. The proximal and distal ends of inner member 72 may be affixed to the hub 56 to the distal tip element 58 by any suitable means, including heat sealing, injection molding, and an adhesive. Of course, inner member 72 may be made of various materials having the desired properties, including stainless steel.

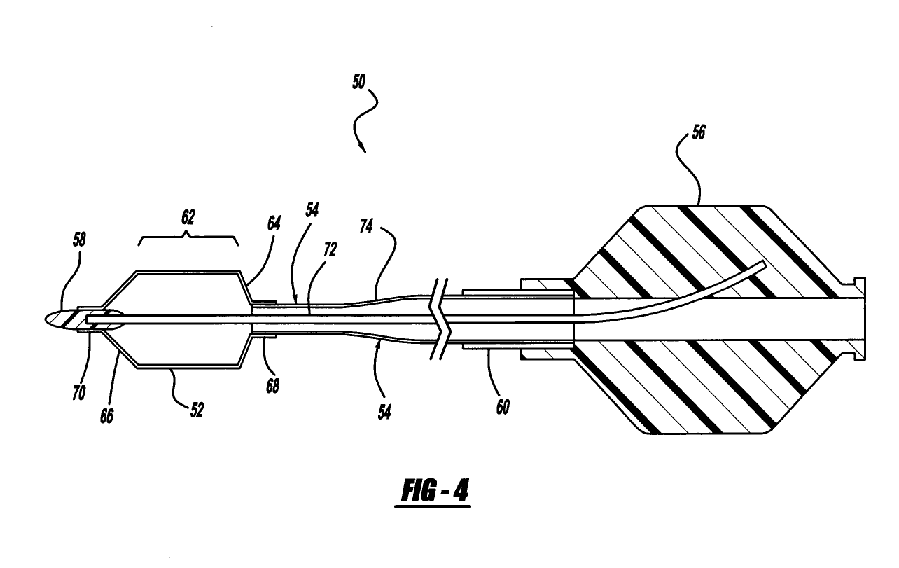

FIGS. 5 and 6 show partially diagrammatic views of a balloon catheter 76 having a similar arrangement, including a balloon 78, a flexible shaft 80, and a hub 82. The shaft 80 in this example has an inner member or stiffening wire 84, and a tubular outer body 86. Outer body 86 also has at least one integral wire 88 extending within the wall of outer body 86, which may be stainless steel, or Kevlar or Dyneema fiber another material having high pull strength and low elongation under stress.

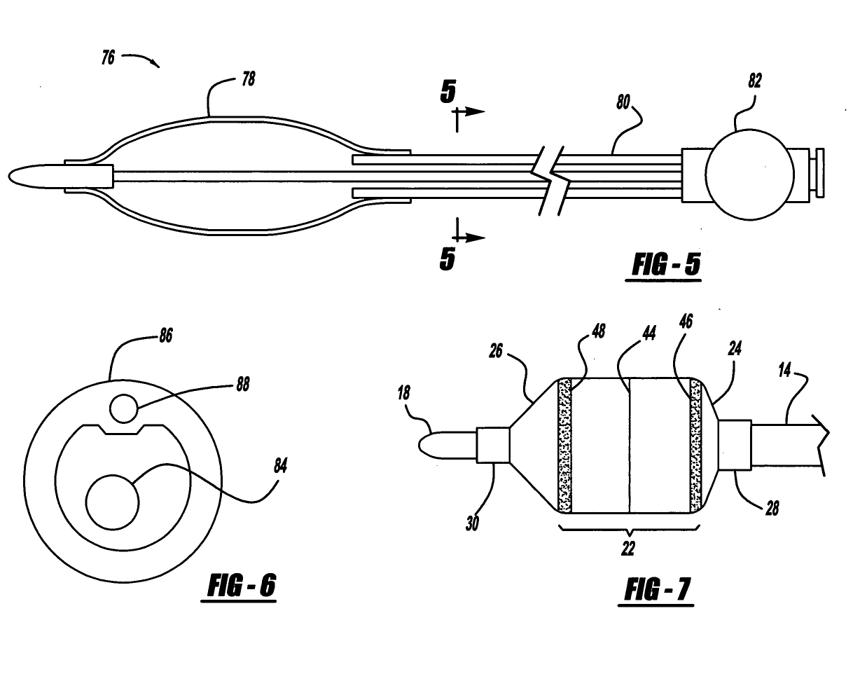

A larger view of a distal segment of a balloon catheter is shown in FIG. 7, including a shaft 14 and distal tip 18, a balloon having a cylindrical working portion 22, and a proximal and distal tapering portion 24 and 26, and proximal and distal legs 28 and 30. The balloon also has a central marker 44 which encircles the longitudinal center of the balloon, as well as a proximal and distal marker 46 and 48 which extend around the circumference of the balloon at the proximal and distal ends of the central working portion 22.

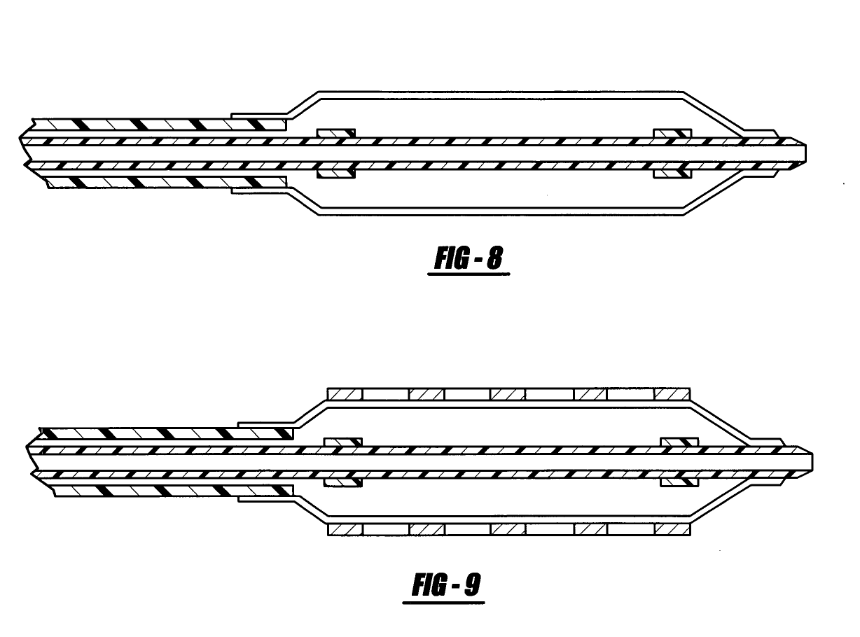

FIGS. 8 and 9 show partial longitudinal cross-sections of a balloon catheter with tubular inner and outer bodies, a balloon, and a pair of conventional radiopaque markers on the inner body. FIG. 9 also shows a stent being deployed by the balloon.

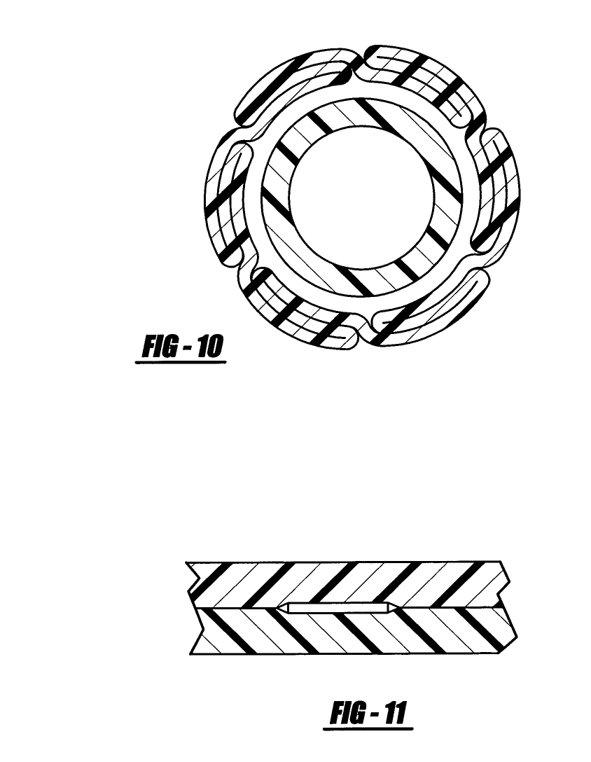

FIG. 10 shows a transverse cross-section of a balloon in an initial configuration. The balloon is deflated, pleated, and wrapped around a catheter shaft tube.

FIG. 11 shows a diagrammatic partial cross-section of a balloon having two layers, and a visual marker imprinted on an intermediate surface between the balloon material layers.

Balloon catheters according to the principles of the present invention may be made of any suitable material using a variety of methods. Various polymers have the desired characteristics of strength, resilience, flexibility, biocompatibility and endurance. Many different materials may be used for manufacturing steerable catheters of the present invention. For example, some of the polymer materials may include polyamides, polyurethanes, nylons, polyethylenes, including high-density polyethylene (HDPE), polyether block amide (PEBA) which is available as Pebax®, polyester (PET), polycarbonate, polypropylene, acrylonitrile-butadiene styrene terpolymer (ABS), or polyetheretherketone (PEEK). Also, any of the catheter components may be made of a co-extrusion or a blend or a block copolymer of such polymer materials.

Many variations on components and designs of a balloon catheter are possible. For example, a reinforcing element may be included using another material, such as Kevlar or Dyneema (HDPE) fibers. Alternately, reinforcing member(s) may be embedded in the wall of the outer body, and may include a single wire or fiber, or may include multiple fibers which may be braided or coiled about the outer body.

It should be understood that an unlimited number of configurations for the present invention could be realized. The foregoing discussion describes merely exemplary embodiments illustrating the principles of the present invention, the scope of which is recited in the following claims. Those skilled in the art will readily recognize from the description, claims, and drawings that numerous changes and modifications can be made without departing from the spirit and scope of the invention.

What is claimed is:

1. A balloon catheter for therapeutically treating a patient, comprising:
a flexible shaft having a proximal end and a distal end; the shaft defining an inflation lumen;
a balloon affixed to the shaft near its distal end; the balloon defining an interior and being made of substantially inelastic balloon material; wherein the balloon is an integral, unitary piece;
the balloon in an initial configuration being deflated, pleated and wrapped around the catheter shaft; and the balloon in an inflated configuration the balloon having a cylindrical working portion extending between a proximal and distal tapering portion, each of which extend to a proximal and distal balloon collar, respectively;
a hub affixed to the proximal end of the shaft and defining at least an inflation port; such that the inflation lumen communicates between the hub inflation port and the balloon interior; and
at least one visual marker imprinted on the balloon material which indicates specific information about at least one attribute of the individual balloon catheter.

2. The balloon Catheter of claim 1, wherein the visual marker indicates the balloon size.

3. The balloon catheter of claim 1, wherein the visual marker characterizes the strength of the balloon.

4. The balloon catheter of claim 1, wherein visual markers characterizes the compliance of the balloon.

5. The balloon catheter of claim 1, wherein at least one marker has a form selected from the group of: one or more lines, dots, characters, letters, numbers, and symbols.

6. The balloon catheter of claim 1, the shaft further comprising an inner member and an outer tubular body; the outer body surrounding at least a portion of the inner member.

7. The balloon catheter of claim 1, wherein the visual marker is color-coded.

8. The balloon catheter of claim 7, wherein the color of the visual marker indicates the nominal diameter size of the balloon, measured at rated burst pressure.

9. The balloon catheter of claim 1, wherein a position of the visual marker is at a longitudinal center of the balloon.

10. The balloon catheter of claim 1, wherein the visual marker is in the form of at least one circumferential line around the balloon.

11. The balloon catheter of claim 1, wherein visual markers are positioned at proximal and distal ends of the cylindrical working portion of the balloon.

12. The balloon catheter of claim 1, wherein visual markers are positioned at a longitudinal center of the balloon, and at proximal and distal ends of the cylindrical working portion of the balloon; and wherein the central visual marker is thinner than the markers at the proximal and distal ends of the cylindrical working portion.

13. The balloon catheter of claim 1, wherein the at least one marker imprinted on an outer surface of the balloon.

14. The balloon catheter of claim 1, wherein the at least one marker is imprinted on an inner surface of the balloon.

15. The balloon catheter of claim 1, wherein the at least one visual marker is a combination marker, viewable with visible light and x-ray fluoroscopy.

16. A balloon catheter for therapeutically treating a patient, comprising:
a flexible shaft having a proximal end and a distal end; the shaft defining an inflation lumen;
a balloon affixed to the shaft near its distal end; the balloon defining an interior and being made of substantially inelastic balloon material; wherein the balloon has more than one layer, and each layer is an integral, unitary piece;
the balloon in an initial configuration being deflated, pleated and wrapped around the catheter shaft; and the balloon in an inflated configuration the balloon having a cylindrical working portion extending between a proximal and distal tapering portion, each of which extend to a proximal and distal balloon collar, respectively;
a hub affixed to the proximal end of the shaft and defining at least an inflation port; such that the inflation lumen communicates between the hub inflation port and the balloon interior; and
at least one visual marker imprinted on an intermediate surface between two of the balloon material layers, which indicates specific information about at least one attribute of the individual balloon catheter.

17. The balloon catheter of claim 16, wherein the at least one visual marker is a combination marker, viewable with visible light and x-ray fluoroscopy.
